(19) United States
(12) Patent Application Publication
(10) Pub. No.: US 2024/0197181 A1
(43) Pub. Date: Jun. 20, 2024

(54) INTRAORAL OPTICAL COHERENCE TOMOGRAPHY SCANNER WITH OPTICAL FIBER ADAPTER

(71) Applicant: Dental Imaging Technologies Corporation, Quakertown, PA (US)

(72) Inventors: Xiaodong TAO, East Rochester, NY (US); Siyu MA, East Rochester, NY (US); Victor C. WONG, Pittsford, NY (US)

(21) Appl. No.: 18/555,155

(22) PCT Filed: Apr. 16, 2022
(86) PCT No.: PCT/US2022/025145

§ 371 Date: Oct. 12, 2023

Related U.S. Application Data
(60) Provisional application No. 63/180,706, filed on Apr. 28, 2021.

(51) Int. Cl. A61B 5/00; A61B 1/07; A61B 1/24

(52) U.S. Cl. CPC A61B 5/0066; A61B 1/07; A61B 1/24; A61B 5/0088

(57) ABSTRACT

An optical coherence tomography scanner for imaging a sample has a wavelength-tunable light source configured to generate scanning light having a range of wavelengths and a scanning probe having a scanning head and one or more optical channels that convey light to and from the scanning head. Each channel has a sample arm with optical fibers for conveying scanning light to the sample and conveying scattered and back-reflected light from the sample to a detector; a reference arm with optical fibers conveying reference light from the wavelength-tunable light source; an optical fiber or fiber system that defines an optical path distance for the sample or reference arm; a detector that generates an output signal according to combined light from the sample arm and conveyed reference light; and a digitizer to generate digital data according to the detector output signal and to communicate the generated digital data to a computer.

### TECHNICAL FIELD

The disclosure relates generally to hand-held, intraoral optical coherence tomography (OCT) imaging and, more particularly, to apparatuses and methods for more compact design of hand-held, intraoral OCT imaging systems.

### BACKGROUND

Optical coherence tomography (OCT) is a non-invasive imaging technique that employs interferometric principles to obtain high resolution, cross-sectional tomographic images that characterize the depth structure of a sample. Particularly suitable for in vivo imaging of human tissue, OCT has shown its usefulness in a range of biomedical research and medical imaging applications, such as in ophthalmology, dermatology, oncology, and other fields, as well as in ear-nose-throat (ENT) and dental imaging.

OCT has been described as a type of “optical ultrasound”, imaging reflected energy from within living tissue to obtain cross-sectional data. In an OCT imaging system, light from a wide-bandwidth source, such as a super luminescent diode (SLD) or other light source, is directed along two different optical paths: a reference arm or path of known optical path length and a sample arm or path that illuminates the tissue or other subject under study. Reflected and back-scattered light from the reference and sample arms is then recombined in the OCT apparatus and interference effects are used to determine characteristics of the surface and near-surface underlying structure of the sample. Interference data can be acquired by rapidly scanning the illumination across the sample. At each of several thousand points along the sample surface, the OCT apparatus obtains an interference profile which can be used to reconstruct an A-scan with an axial depth into the material that is largely a factor of light source coherence. For most tissue imaging applications, OCT uses broadband illumination sources and can provide image content at depths of up to a few millimeters (mm).

There are significant limitations to the various techniques and approaches that have been applied to the problems of intraoral imaging. Constraints on camera and scanner size and form factor and the confined space requirements of the intraoral imaging environment make it challenging to accurately characterize intraoral surfaces. It can be difficult to focus with accuracy on individual surface features, to provide image content of broad areas of patient dentition at suitable resolution and focus, and to provide sufficient illumination for diagnostic purposes.

Accurate imaging of the tooth and other intraoral structures can be compromised due to the effects of fluids. Water, saliva, blood, and other fluids that can collect on and around the teeth can cause difficulties for OCT as well as for reflective imaging systems. For some illumination apparatus, only a portion of the projected light impinges onto the tooth surface, sample S. Similarly, the backscattered light from the tooth surface is again refracted at the fluid-air interface and captured by a camera at another angle. Back-ray tracing of the projection beam and captured light beam locates an intersection point which is shifted toward the imaging system, causing image distortion.

In addition to dimensional inaccuracy, reflection from fluids in the mouth can produce shining spots on images, saturated due to high reflection levels. Still other problems that can be particularly pronounced for intraoral imaging include tight space constraints, fogging, wetness from blood/saliva/water, translucency of teeth, high levels of light absorption and scattering by gum/cheek/tongue, and patient gag reflex, for example. For reasons such as these, intraoral imaging presents considerable challenges over and above problems encountered with most other biomedical imaging applications, with respect to operation, environment, and image quality.

Conventional OCT systems adapt the architecture of an interferometer, which typically consists of both fiber-based and free-space optics and mechanical components, such as one or more fiber couplers, fiber circulators, lenses, and mirrors, for directing light to and from its sampling and reference arms. Acquiring reliable and accurate interference signals requires precision matching of the sampling and reference arm optical paths. An adjustable mechanical reference arm is often used for achieving the optimal optical path length. This requirement, however, presents some inherent difficulties, including the following:
(i) added size, weight, and cost to the scanning apparatus. An adjustable mechanical reference arm often employs multiple optical mounts, translation stages, kinematic mounts, and optics components. To prevent the contamination of mirror or lens elements, the optical system often needs a specially designed enclosure, adding further cost, weight, and bulk to the system and making it poorly adapted to a clinical or dental chair setting.
(ii) sensitivity to vibration and mechanical drift. Because the reference arm often includes a number of components including a free space fiber coupler, it can be very sensitive to environmental vibration. Temperature change may also cause mechanical drift in the reference arm, potentially compromising image quality.
(iii) high maintenance and downtime costs. To maintain high fiber coupling efficiency, regular realignment is often required, such as on a yearly or monthly basis. This type of adjustment often needs to be performed by specialists, which increases maintenance cost and downtime cost.
(iv) increased cost of manufacture. To provide high fiber coupling efficiency, special adjustments need to be performed for tuning the reference arm, extending the time needed for final assembly and test.
(v) high insertion loss of the reference arm. The conventional free-space reference arm often has higher insertion loss because of the low coupling efficiency of the free space fiber coupler.

These problems become increasingly more complex for an OCT scanning device that uses multiple scanning channels. Improvements that reduce size and cost and help to eliminate sources of mechanical drift and sensitivity would be beneficial for making OCT imaging more usable, robust, and affordable.

### SUMMARY

An object of the present disclosure is to advance the art of intraoral OCT imaging. An embodiment of the present disclosure particularly addresses the need for improved methods for adjusting the optical path length for OCT interferometer components.

Another object of this application is to address, in whole or in part, at least the foregoing and other deficiencies in the related art.

It is a related object of this application to provide, in whole or in part, at least the advantages described herein.

These objects are given only by way of illustrative example, and such objects may be exemplary of one or more embodiments of the application. Other desirable objectives and advantages inherently achieved by the disclosed methods may occur or become apparent to those skilled in the art. The invention is defined by the appended claims.

According to one aspect of the disclosure, there is provided an optical coherence tomography scanner for imaging a sample, the optical coherence tomography scanner comprising:
a) a wavelength-tunable light source configured to generate scanning light having a range of wavelengths;
b) a scanning probe having a scanning head and one or more optical channels that convey light to and from the scanning head, each channel comprising:
   (i) a sample arm comprising optical fibers for conveying the scanning light to the sample and conveying scattered and back-reflected light from the sample to a detector;
   (ii) a reference arm comprising optical fibers for conveying reference light from the wavelength-tunable light source;
   (iii) an optical fiber or fiber system that defines an optical path distance for the sample arm or the reference arm;
   (iv) a detector that generates an output signal according to combined light from the sample arm and the conveyed reference light; and
c) a digitizer that is energizable to generate digital data according to the detector output signal and to communicate the generated digital data to a computer for storage or display.

### BRIEF DESCRIPTION OF THE DRAWINGS

The foregoing and other objects, features, and advantages of the invention will be apparent from the following more particular description of the embodiments of the disclosure, as illustrated in the accompanying drawings.

The elements of the drawings are not necessarily to scale relative to each other.

FIG. 1 is a schematic diagram showing an example swept-source OCT (SS-OCT) apparatus according to an embodiment of the present disclosure.

FIG. 2A shows a schematic representation of scanning operation for obtaining a B-scan.

FIG. 2B shows an OCT scanning pattern for C-scan acquisition.

FIG. 3A is a schematic diagram that shows a high-speed intraoral OCT system of the present disclosure having multiple channels.

FIG. 3B is a schematic diagram that shows components that collimate, focus, and scan light from each channel.

FIG. 3C is a schematic diagram showing a channel with an additional camera for viewing the imaged sample.

FIG. 4A shows a schematic for an apparatus using a one-dimensional array for providing output beams from multiple channels.

FIG. 4B shows a schematic for an apparatus using a two-dimensional array for providing output beams from multiple channels.

FIG. 5 is a schematic diagram showing an apparatus for scanning multiple channels at different depths.

FIG. 6 is a schematic diagram showing an apparatus for scanning multiple channels with different optical lengths for each sample arm.

FIG. 7 is a schematic diagram that shows use of a fiber array and optical switching for scanning a region of interest.

FIG. 8A is a schematic diagram that shows an OCT scanner having a fiber reference arm.

FIG. 8B is a schematic diagram that shows an OCT scanner having a fiber reference arm in an alternate embodiment.

FIG. 9 is a schematic diagram that shows use of fiber adapters in a multi-channel system.

FIG. 10A shows an optical fiber that provides the reference arm for OCT imaging.

FIG. 10B shows an embodiment that uses a fiber ferrule for defining the reference arm OPD.

FIG. 10C shows use of a sleeved arrangement with two ferrules.

FIG. 10D shows a method of fiber tapering for adjusting the OPD.

FIG. 10E shows tapering for an embodiment not using connectors or ferrules.

FIG. 10F shows an embodiment using a fiber stretcher for adjusting the OPD.

FIG. 11A is a schematic view showing an OCT system with probe and external laser, detector, and digitizer.

FIG. 11B is a schematic view showing an OCT system with probe housing the detector and with external laser and digitizer.

FIG. 11C is a schematic view showing an OCT system with probe housing the detector, and digitizer and having an external laser.

FIG. 11D is a schematic view showing an OCT system with probe housing the laser, detector, and digitizer.

### DESCRIPTION OF EXAMPLE EMBODIMENTS

The following is a detailed description of example embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

The general term “scanner” relates to an optical system that is energizable to project a scanned light beam of light, such as broadband near-IR (BNIR) light that is directed to the tooth surface through a sample arm and acquired, as reflected and scattered light returned in the sample arm, for measuring interference with light from a reference arm used in OCT imaging of a surface. The term “scanner” can also refer to a scanning optical element, such as an actuable MEMS (micro-electromechanical systems) scanner, such as an actuable MEMS mirror or mirror array, for example. The term “raster scanner” relates to the combination of hardware components that sequentially scan light toward uniformly spaced locations along a sample, as described in more detail subsequently.

In the context of the present disclosure, the phrase “imaging range” relates to the effective distance (generally considered in the z-axis or A-scan direction) over which OCT measurement is available. The OCT beam is considered to be within focus over the imaging range. Image depth relates to imaging range, but has additional factors related to signal penetration through the sample tooth or other tissue.

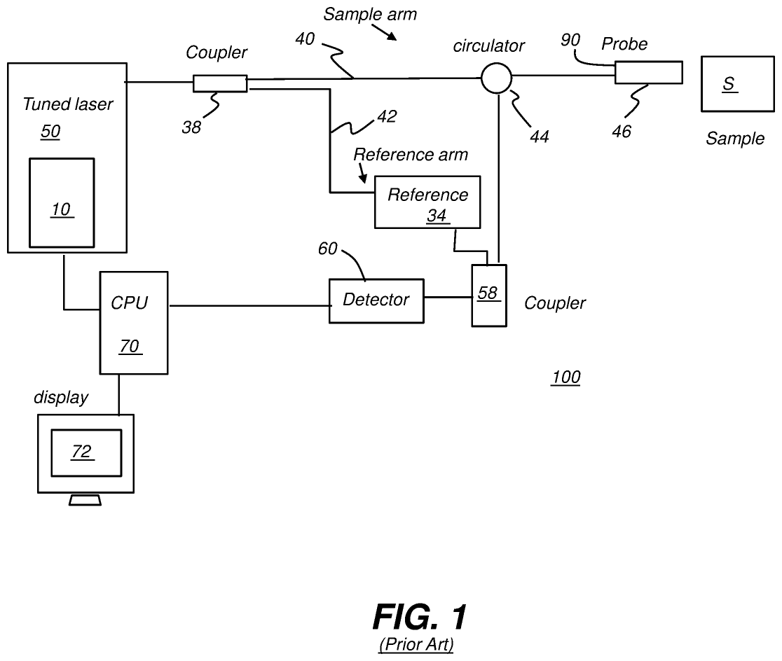

By way of example, the simplified schematic diagram of FIG. 1 shows the components of one type of OCT apparatus, here, a conventional swept-source OCT (SS-OCT) apparatus 100 using a Mach-Zehnder interferometer (MZI) system with a wavelength-tunable light source provided by a wavelength filter 10 that is part of a tuned laser source 50, which can be a laser, super-luminescent light-emitting diode (LED), super-continuum light source, or other type of wide-bandwidth light source. For intraoral OCT, for example, laser 50 can be tunable over a range of frequencies (expressed in terms of wave-numbers k) corresponding to wavelengths between about 400 and 1600 nm. According to an embodiment of the present disclosure, a tunable range of about 60 nm bandwidth centered about 1300 nm is used for intraoral OCT.

In the FIG. 1 device, the variable tuned laser 50 output goes through a coupler 38 and to a sample arm 40 and a reference arm 42. The sample arm 40 signal goes through a circulator 44 and is directed for imaging of a sample S from a handpiece or probe 46. The sampled signal is directed back through circulator 44 and to a detector 60 through a coupler 58. The reference arm 42 signal is directed by a reference 34, which can be a mirror or a light guide, through coupler 58 to detector 60. The detector 60 may use a pair of balanced photodetectors configured to cancel common mode noise.

Control logic processor (control processing unit CPU) 70 is in signal communication with tuned laser 50 and its programmable filter 10 and with detector 60. Processor 70 can control the scanning function of probe 46 and store any needed calibration data for obtaining a linear response to scan signals. Processor 70 obtains and processes the output from detector 60. CPU 70 is also in signal communication with a display 72 for command entry and OCT results display.

It should be noted that the swept-source architecture of FIG. 1 is one example configuration only; there are a number of ways in which the interferometer components could be arranged for providing swept-source OCT imaging.

Among proposed strategies for obtaining higher image acquisition speeds in an OCT system is simply using a high sweep-rate wavelength-tunable light source. However, as previously observed in the background section, the problem is more complex; attempts to operate at faster sweep rates have led to increased cost and can yield disappointing results with regards to the diagnostic benefits and overall quality of the OCT image content.

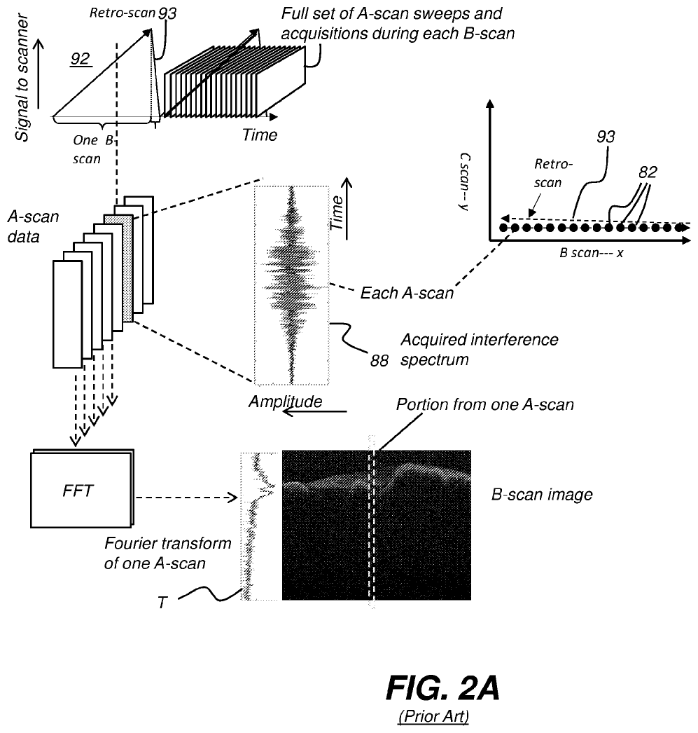

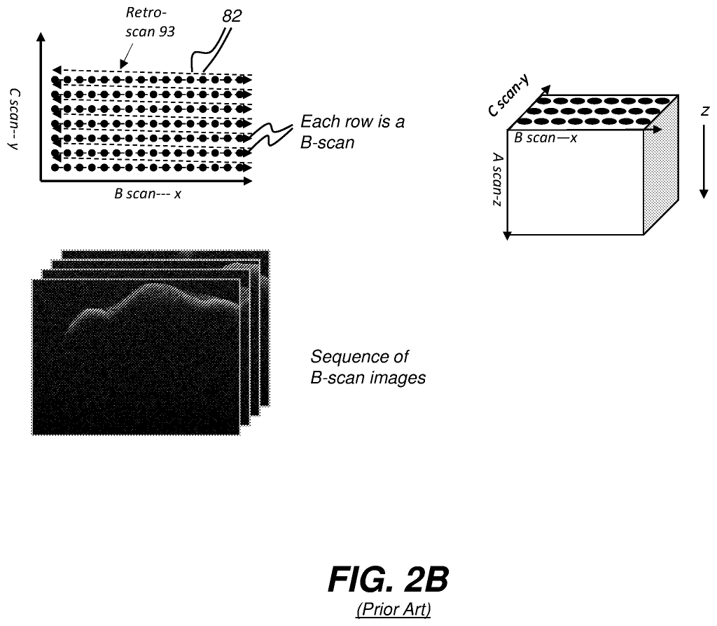

By way of further background, FIGS. 2A and 2B give an overview of the OCT scanning pattern as executed by probe 46. At each point in the scanning sequence, the OCT device performs an A-scan. A linear succession of A-scans then forms a B-scan, corresponding to the x-axis direction as shown. Successive B-scan rows, side-by-side, then form a C-scan which provides the 3D OCT image content for the sample S.

FIG. 2A schematically shows the information acquired during each A-scan. The scan signal for obtaining each B-scan image has two linear sections in the example shown, with a scan portion 92, during which the scanning mirror is driven to direct the sampling beam from a beginning to an ending position, and a retro-scan 93, during which the scanning mirror is restored to its beginning position. An interference signal 88, shown with DC signal content removed, is acquired over the time interval for each point 82, wherein the signal is a function of the time interval required for the sweep, with the signal that is acquired indicative of the spectral interference fringes generated by combining the light from reference and feedback sample arms of the interferometer (FIG. 1). The Fourier transform FFT generates a transform T for each A-scan. One transform signal corresponding to an A-scan is shown by way of example in FIG. 2A.

From the above description, it can be appreciated that a significant amount of data is acquired over a single B-scan sequence. In order to process this data efficiently, a Fast-Fourier Transform (FFT) is used, transforming the time-based signal data to corresponding frequency-based data from which image content can more readily be generated.

In Fourier domain OCT, the A scan corresponds to one line of spectrum acquisition which generates a line of depth (z-axis) resolved OCT signal. The B scan data generates a 2-D OCT image along the corresponding scanned line.

Raster scanning is used to obtain multiple B-scan data by incrementing the raster scanner 90 acquisition in the C-scan (y-axis) direction. This is represented schematically in FIG. 2B, which shows how 3-D volume information is generated using the A-, B-, and C-scan data.

The wavelength or frequency sweep sequence that is used at each A-scan point 82 can be modified from the ascending or descending wavelength sequence that is typically used. Arbitrary wavelength sequencing can alternately be used. In the case of arbitrary wavelength sequencing, which may be useful for some particular implementations of OCT, only a portion of the available wavelengths are provided as a result of each sweep. In arbitrary wavelength sequencing, each wavelength can be randomly selected, in arbitrary sequential order, to be used in the OCT system during a single sweep. A-scan points 82 can be uniformly spaced from each other with respect to the x axis, providing a substantially equal x-axis distance between adjacent points 82 along any B-scan image. Similarly, the distance between lines of scan points 82 for each B scan can be uniform with respect to the y axis. X-axis spacing may differ from y-axis spacing; alternately, spacing along these orthogonal axes of the scanned surface may be equal.

For conventional OCT approaches, image acquisition speed is related to factors of sweep rate and digitizer capability. Faster sweep rates can, in turn, allow improved A-scan frequencies, but at the cost of higher noise. High-speed digitization components are also needed at higher acquisition rates, with significant increase in component cost for the needed performance. Thus, there are some practical limits to scanning speed and overall OCT performance that can limit the use of OCT for chairside diagnosis and treatment.

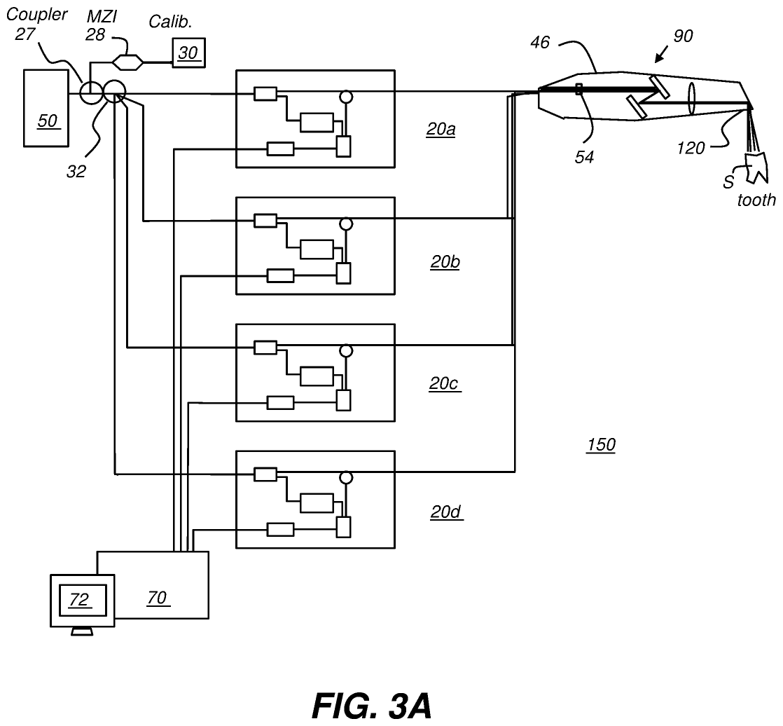

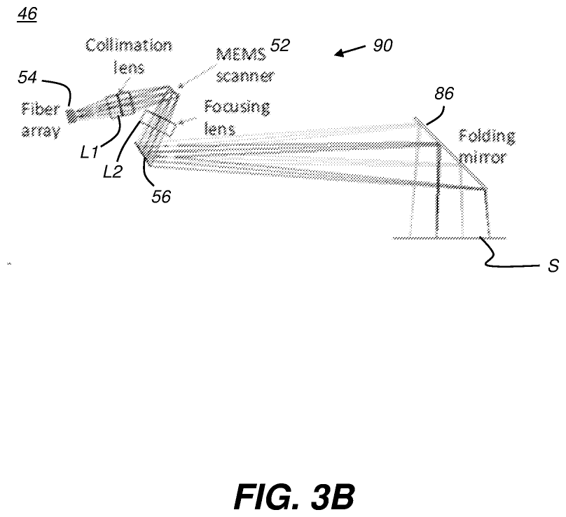

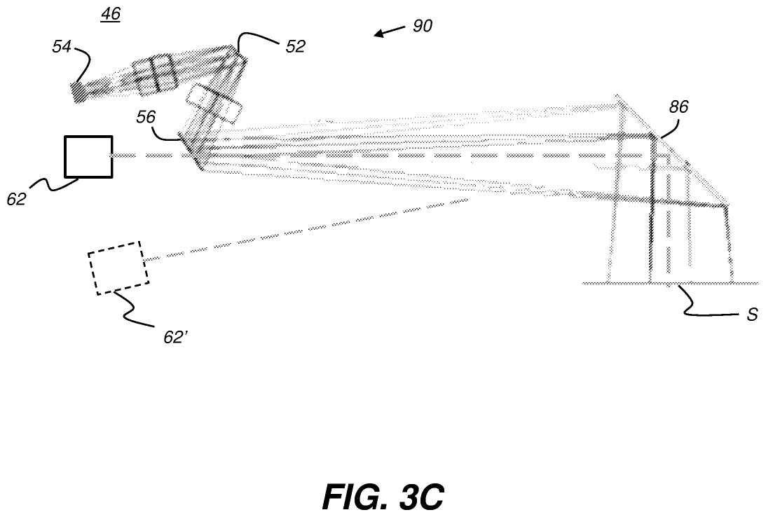

An embodiment of the present disclosure, shown schematically in FIG. 3A, addresses problems of image acquisition speed and the need for increased imaging range by using a multi-channel approach to dental OCT scanning and data acquisition. Referring to the schematic diagram of FIG. 3A, there is shown an example high-speed intraoral OCT system 150 of the present disclosure having multiple that share a common scanning head. For increasing amounts of scanning speed, the number of channels N can be two, three, or four, such as the four channels 20a, 20b, 20c, and 20d that lead to a scanning head 120 as shown in FIG. 3A. Additionally, five or more channels could be used, following the overall pattern described for four channels herein. The scanner 90 within probe 46 directs light originating from swept-wavelength laser source 50 in multiple channels to the tooth or other sample S.

As illustrated in FIG. 3A, a fiber coupler 27 splits off a small portion of the laser light to a Mach-Zehnder interferometer MZI 28. The interference light from the MZI is collected by a photodetector and additional circuit 30 to provide K-clock (K-trigger) signals, which are timing control triggers having equal wavenumber spacing defined in time. Given equal spacing of these signals, the OCT signal sampled with the K-clock timing is linear in wavenumber space. Alternately, the OCT signal can be resampled into a linear wavenumber space using the interference signal from MZI 28. (Zero crossing of the Mach-Zehnder interference (MZI) signal can be used to generate K-trigger signals to prompt the acquisition of SS-OCT signals.) The bulk of the swept-source laser 50 light output is fed into the multi-channel system for OCT imaging through a splitter 32, such as a PLC (Planar Lightwave Circuit) splitter. In each channel, the light illuminates a fiber optic interferometer that has a circulator 44 and a 90/10 fiber coupler 38 that splits light into reference and sample arms 42, 40 (FIG. 1). The system can optionally include additional detectors and optical components to provide polarization sensitive optical coherence tomography. Each channel directs light through probe 46 to a scanning head 120.

FIG. 3B shows probe 46 components that collimate, focus, and scan light from each of the four channels 20a, 20b, 20c, and 20d. As shown in the schematic of FIG. 3B, the multi-channel sampling arms are connected with a fiber array 54 inside of the scanner handpiece, probe 46, which can be used for intraoral or extraoral imaging. Connection of the variable wavelength light can be via a ribbon fiber (not shown). The fiber array 54 aligns the optical fiber cores precisely with desired pitch. The light from the fiber array goes through a collimation lens L1 and to a MEMS (micro-electromechanical systems) scanner 52. Scanned light then goes through a focusing lens L2 as shown in FIG. 3B. This focused light reflects from a first folding mirror surface 56 and a second folding mirror surface 86 and is directed to sample S. Multiple spots are focused on the sample S surface with desired spacing; each spot is from one of the multiple channels 20a, 20b, 20c, and 20d.

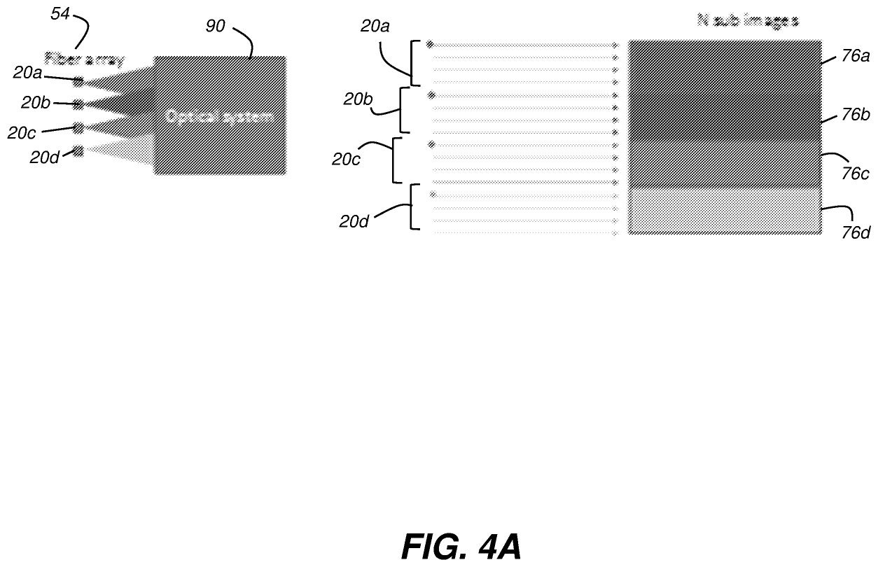

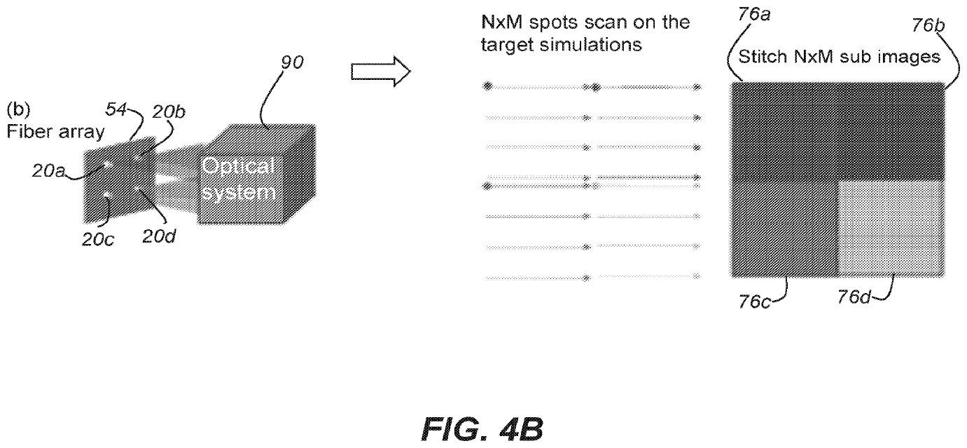

As is shown in the schematic of FIG. 3C, probe 46 can optionally include other components, such as a camera 62 for obtaining color information or to assist in probe movement, for example. Where camera 62 is used, surface 56 can be a dichroic surface, treated to reflect the IR light used for OCT scanning and to transmit visible light to the camera 62. A camera can alternately be provided at an oblique angle with respect to optical axis OA; by way of example, an alternate position of a camera 62′, which can be a second camera or the only camera, is shown in FIG. 4.

Fiber array 54 within probe 46 can have a number of different configurations. FIG. 4A shows fiber array 54 arranged in line as a one-dimensional (1D) array that simultaneously provides an output beam from each channel 20a, 20b, 20c, and 20d. The 1D array configuration can be used to direct the scanned beams to multiple spots, aligned on the target sample S. Scanning of a number N of illumination beams in this manner can be used to generate a number N of adjacent sub-images, shown as sub-images 76a, 76b, 76c, and 76d in the four-channel example of FIG. 4A. Processing software can then be used to stitch together the N adjacent images that lie along the scan line.

In scanning with a one-dimensional optical array using the FIG. 4A arrangement, the field of view (FOV) is divided in number of strips. Each focused spot from a channel scans only a small sub-region of the FOV. The reflected light from each focused spot at the sample is collected by probe 46 optics and is guided to the sampling arms of each channel. Light beams from the sample and reference arms 40 and 42 (FIG. 1) are recombined in the detection arms through a 50/50 coupler 58. Interference fringes that are formed are detected by balanced photo detectors or other mechanism in detector 60. The analogue signal from the balanced photo detector 60 can be digitized by a data acquisition card. The image volume from each channel can be generated using an OCT reconstruction algorithm. Finally, a reconstruction of the complete scanned image volume can be formed by stitching together the different sub-image volumes.

FIG. 4B shows an alternate arrangement using a 2×2 fiber array 54 to scan the FOV. This arrangement generates sub-image content as an array of images for stitching.

Since each channel scans only part of the field of view, the multi-channel system can achieve a much faster speed as compared to a single channel system. Using N multiple channels, scanning simultaneously, the complete FOV can be scanned in a fraction 1/N of the time required for the conventional single-channel arrangement.

Because the source laser output is split between N channels, some increase in laser power is needed in order to provide multi-channel OCT imaging capability. According to an embodiment of the present disclosure, a 40 mW laser is used to drive four channels, with output power subdivided to provide 10 mW in each channel.

In general, to achieve the same scanning speed, the swept laser source in an N-channel system only requires 1/N the sweep rate used in a single channel system. Lowering of the sweep rate accordingly lowers the digitization speed requirement of the data acquisition card, which can dramatically reduce the system cost.

To achieve the same imaging range, the frequency of the OCT signal, f_{OCT}, can be much lower with the multi-channel system than the frequency used in a single channel system. f_{OCT} may be expressed as follows:

$${f}_{OCT} = \frac{{f}_{s} Δ λ Z}{{αλ}^{2}} ,$$

wherein: Δλ is the bandwidth of the laser spectrum;
λ is the central wavelength;
Z is the imaging range;
a is the duty cycle of the laser; and
fₛ is the frequency of the swept laser source.

Since, in an N-channel system, the frequency of the OCT signal is only 1/N of the frequency used in a single channel system, the digitizer can operate at a lower sampling rate. Thus, N-channel design can reduce both cost and system noise. Alternatively, if the same high-speed digitizer that is used for a single scanner OCT probe is used in an N-channel system, performance can be improved, at up to N times of the imaging range.

### Variable Range Scanning

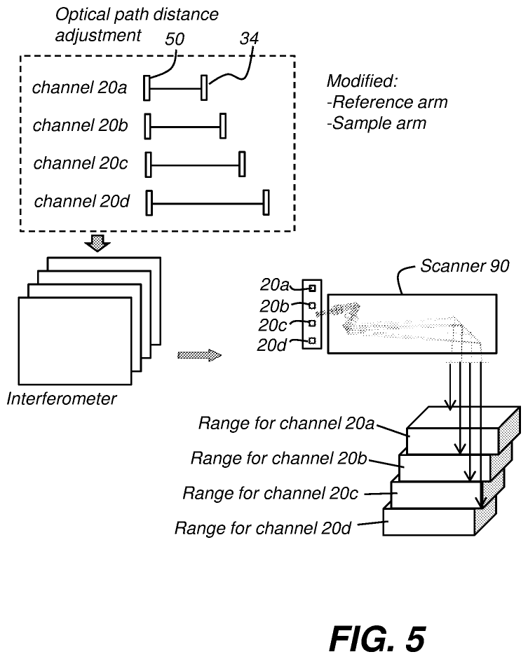

The multi-channel system also has the ability to extend the effective imaging range of the scanner without impact on the sampling rate. By introducing additional optical path difference (OPD) in the reference arm or the sampling arm, the beam from each channel can scan a different range of the target as shown schematically in FIG. 5. The range can be extended by factor of N, when an N channel system is used. However, this configuration may reduce the scanning speed over other arrangements, since each channel needs to scan the whole field of view.

By simultaneously scanning N channels and using image processing to stitch together the image content of the individual channels, embodiments of the present disclosure can process the corresponding image content in parallel and significantly reduce the overall scan time needed for OCT imaging over a given sample region and at desired scanning range.

Simultaneous multichannel scanning, with each channel scanning at a different range, effectively expands the overall imaging range available from the OCT scanner. The scanning arrangement of FIG. 5 shows schematically how variable range within a channel can be achieved within the interferometry subsystem for the channel, according to an embodiment of the present disclosure. By varying the relative optical path lengths of reference and sample arms or paths 42 and 40, respectively, in each channel (FIG. 1), the scanned range in the z-direction for each individual channel can be modified.

Within the interferometry system for each channel, the reference arm 42 typically includes some type of mirror or other reflective surface. The distance that light travels towards and back from the reflective surface, that is, the optical path delay for the reference arm, directly relates to a particular range within the sampled material. Thus, by adjusting the optical distance between the reflective or back-scattering material and interferometry combining components, returned light from variable depths within the sample contributes to the detection signal.

An alternate approach for scanning at different range, not shown in FIG. 5, changes the optical path delay of the sampling arm for each channel.

Methods for changing the optical path delay can include adding a length of optical fiber between two points along the light path, adding an optical stretcher, or adding a variable fiber delay line using a fiber collimator and movable reflectors or fiber stretcher, or adding light guides or other transmissive features of higher or lower refractive index into the light path.

### Adding Optical Switching

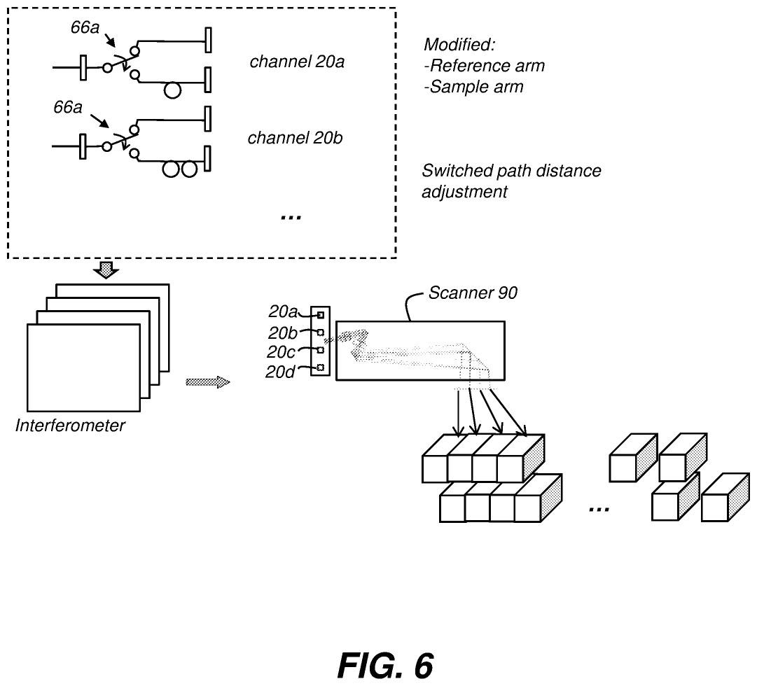

FIG. 6 displays a flexible way to extend the imaging range and to obtain various scanning patterns by adding an optical switch to each channel, wherein the optical switch selects alternate light paths of different optical path length. For the sake of example, two optical switches 66a and 66b for two channels 20a and 20b are shown; additional channels in an N-channel configuration could also be switched following the same pattern. It can also be noted that different switched patterns can be used to simultaneously scan different areas and different ranges using a swept-scan laser signal according to embodiments of the present disclosure. Thus, in the four-channel configuration schematically represented in FIG. 6, each channel can be switched to scan to a first range over its target sample region. The switching arrangement can then be changed to scan to a second range over the corresponding area of the sample. Multiple switch positions can be provided for each channel, allowing multiple optical path delays for any one or more channels and, as a result, multiple scan ranges. This sequence can achieve a large and adaptive imaging range with minimal motion artifacts.

It can readily be seen that using a switched delay arrangement with multiple scanning channels as represented in FIG. 6 allows the OCT scanning apparatus to extend and adapt imaging range without sacrificing scanning speed. Implementation of variable-range scanning can also be used to accommodate variables in surface contour, such as abrupt transitions in shape and contour characteristic of teeth and other intraoral features. A high-speed switcher can readily change the range settings between two or more scanning volumes, which provides the capability for real-time range adaptation.

### ROI Scanning

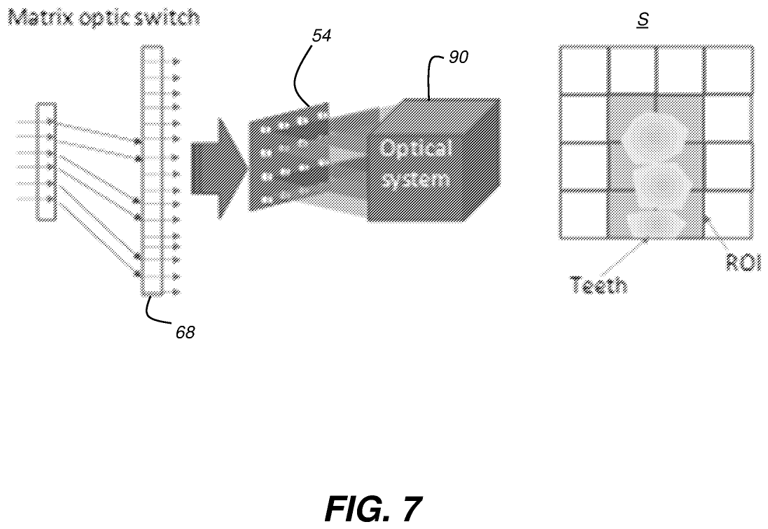

The multi-channel OCT system can also provide the option of adaptive region of interest (ROI) scanning. FIG. 7 illustrates a configuration for such ROI scanning, wherein a matrix optical switch 68 and a 2-D fiber array 54 are integrated with the scanner system. Using matrix switch 68 capabilities, incoming light from multiple channels is redistributed to multiple sub-regions in the FOV. The combined sub-regions define a region of interest (ROI) within the field of view. This configuration can effectively use the light to image a particular feature of interest at high speed. The capability to selectively shape the scanned region can dramatically reduce the volume of the data acquired for reconstruction and storage.

Additionally, by combining ROI selectivity with adjustable range scanning, as described previously with respect to FIGS. 5 and 6, embodiments of the present disclosure can help to provide highly accurate OCT imaging results as the intraoral surface is scanned, without requiring significant computational resources and time.

To address problems of cost, footprint, complexity, reliability of reference arm adjustment and tuning, and high insertion loss of reference arm, as noted previously in the background section, an embodiment of the present disclosure provides an alternative all-fiber configuration. The all-fiber configuration defines the reference optical path distance solely by light conveyed within optical fiber. In contrast to conventional interferometry design, no mirror or other reference reflected surface is needed; the optical path distance defined by the optical fiber serves to provide the reference signal. In addition to advantages related to size, weight, cost, and complexity, the Applicant's approach can also help to provide improved stability, reducing channel drift and enhancing image quality for OCT scanner image acquisition in multi-channel as well as in single-channel embodiments.

The Applicant's approach simplifies the tuning process for the reference arm by using optical fiber that is configured for the optical path distance that is needed, substituting an optical fiber for the conventional mechanical adjustment devices that have been used. The use of a properly coupled optical fiber is made possible using one of a set of pre-fabricated fiber adapters, selected and optimized according to optical path length.

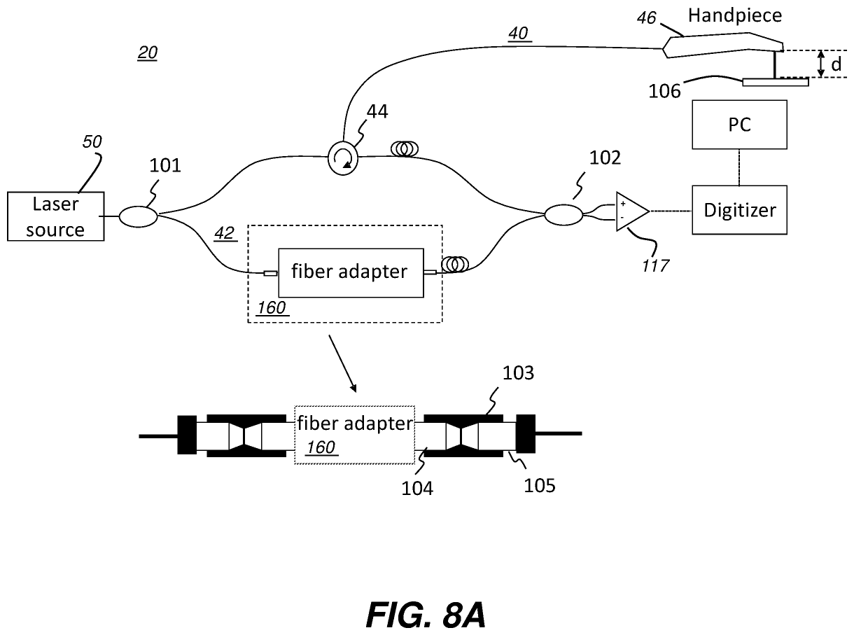

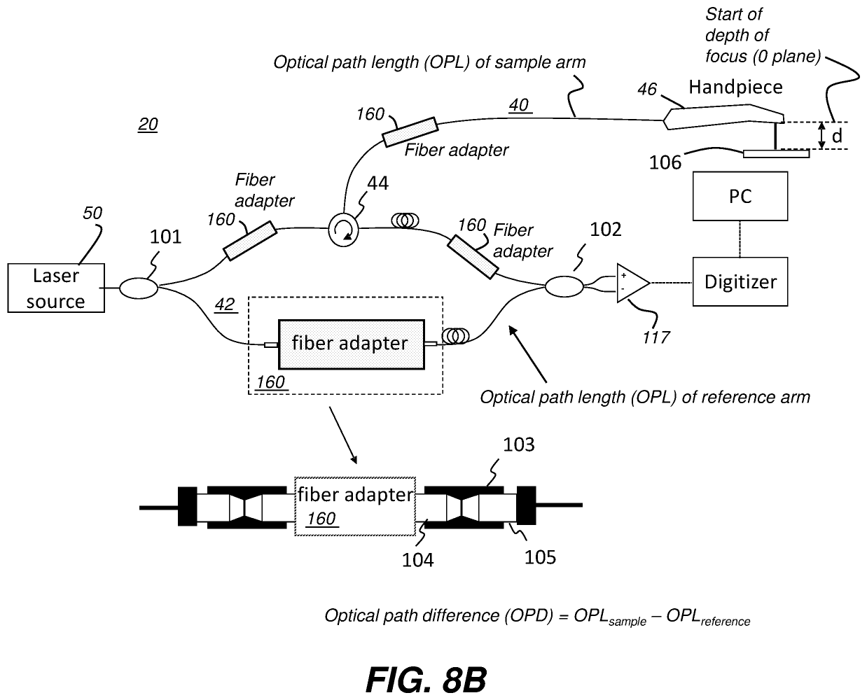

FIG. 8A shows components of a single-channel OCT scanner 20 according to an embodiment. Specification of optical fiber length can be provided using a calibration arrangement through the same channel with probe 46 directed, over a known distance d, to a sample 106 that is a flat mirror or other highly reflective flat reference surface. Referring to the schematic diagram of FIG. 8A, OCT channel 20 has a splitter 101 that directs light from laser source 50 into sampling arm 40 and reference arm 42. Circulator 44 directs light to a handpiece, probe 46, which collects scattered light from the sample and directs collected light through a splitter 102 for interference with light from reference arm 42 and sensing at a balanced detector 117, as shown. Reference arm 42 includes a fiber adapter 160 that is configured for optical path distance matching between reference arm 42 and sample arm 40, so as to achieve a zero optical path length difference (OPD). Fiber adapter 160 has a length of optical fiber that is selected for providing the correct OPD; in practice, a standard set of pre-fabricated fiber adapters 160 can be provided, enabling test personnel to measure and test each of one or more fiber lengths in order to identify the best candidate for installation into a particular scanning device. Alternately, a specific fiber length can be cut. In the embodiment shown in FIG. 8A, the fiber interferometer consisting of the reference arm 42 and sample arm 40 is external to handpiece 46. The start of depth of focus, or zero (0) plane, is at the handpiece output as shown.

According to an embodiment of the present disclosure, the complete reference arm 42 and sample arm 40 can be packaged within handpiece 46; alternately, significant portions of OCT channel 20 can be housed within handpiece 46, as described in more detail subsequently.

Cutting the optical fiber to a certain length can provide a type of “coarse” adjustment for optical path distance. An embodiment of the present disclosure can then provide additional fine-tuning adjustment for OPD as part of fiber adapter 160.

As is further shown in FIG. 8A, fiber adapter 160 has one or more optical connectors 104 with a sleeve 103 for protecting the connection with mating reference arm connectors 105.

Fiber adapter 160 for each channel can be specified and tested as part of manufacture/final assembly. According to an embodiment, a pre-fabricated fiber adapter 160 of a known refractive index is installed on the reference arm 42 for the channel. A test target, such as a flat mirror, can then be scanned at a specified distance from the handpiece. By measuring the location of the mirror response in the OCT signal, the optical path length information can be obtained with a well calibrated system. With the information of refractive index for the optical fiber, the amount of needed adjustment can be calculated. The optical path length information that is obtained can then be used to calculate an amount of needed adjustment. Incremental adjustment for optical path length matching can be available using an adjustment screw or other feature on fiber adapter 160. The desired OPD can be achieved by the adjustment based on calculated OPD. Alternately, the adjustment can be performed manually or automatically while monitoring the OCT signal in real time.

OCT signals, or other interferometry signals, can be analyzed to determine the OPD between sample and reference arms so that an optimal reference distance can be calculated. The installed fiber adapter 160 can then be adjusted as needed in order to provide the proper optical path length. Alternately, an adjusted adapter 160 with slightly longer or shorter length can be fabricated, installed, tested, and adjusted in order to provide the needed optical pathlength for the reference arm. Testing and adjustment can be repeated until results are within acceptable tolerance.

In addition to the reference arm, fiber adapter 160 can be used in other portions of the OCT system, as shown in the example of FIG. 8B that employs multiple fiber adapters. For example, fiber adapter 160 can also be used in the sample arm 40. In practice, fiber adapter 160 can be in at least one or a combination of the places shown.

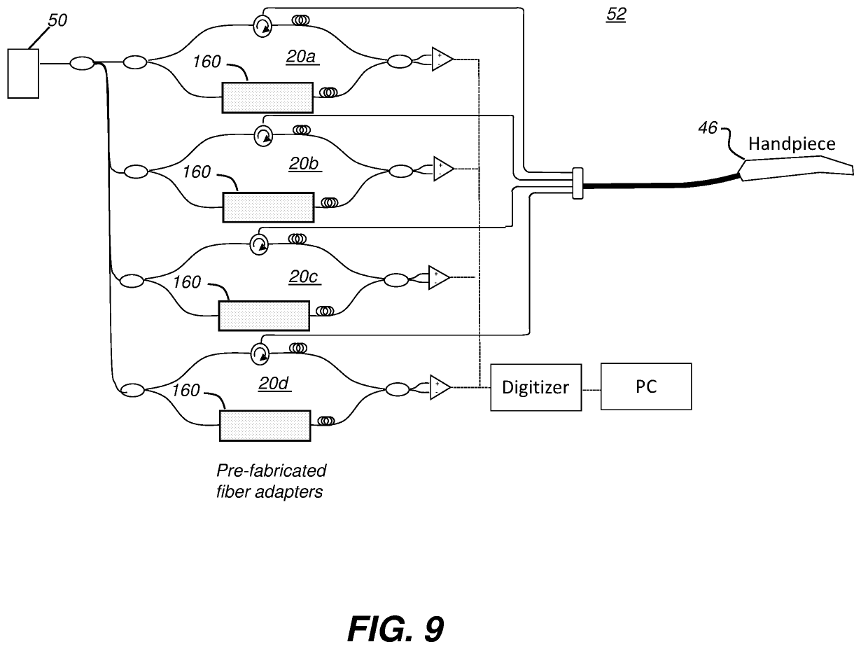

The schematic diagram of FIG. 9 shows use of fiber adapters 160, one for each channel 20a, 20b, 20c, 20d, with a multichannel OCT scanner 52. In this multichannel embodiment, the interferometer components for each channel are shown to be external to the probe 46; preferably, the optical fiber components are housed within probe 46.

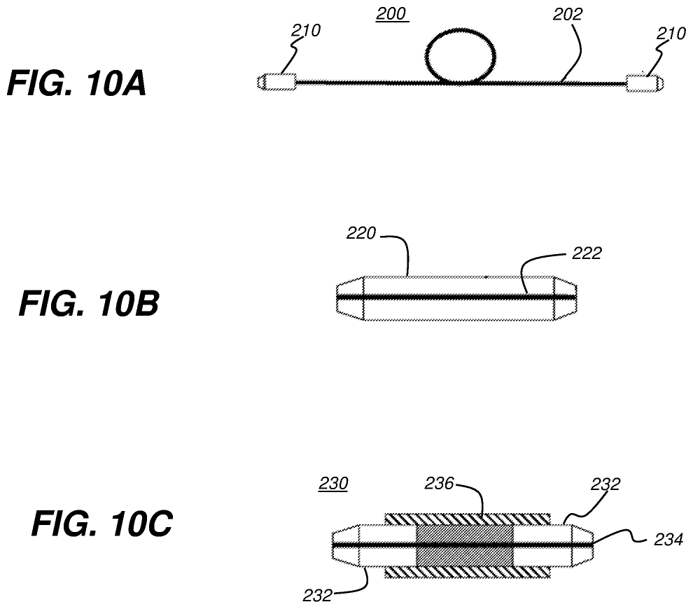

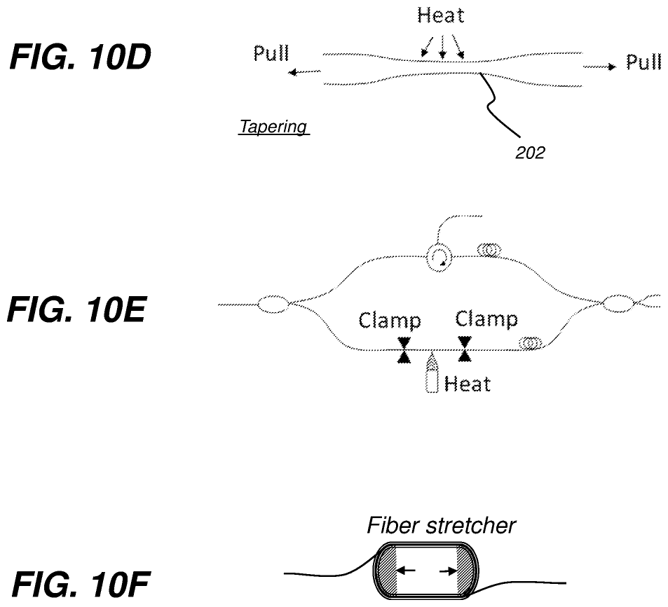

FIGS. 10A-10F show a number of fiber adapter arrangements and techniques for providing fiber adapter 160 that can help to provide reference arm 42 or sample arm 40 with the correct optical path length. FIG. 10A shows an optical fiber assembly 200 with a segment of optical fiber 202 of appropriate length to match the optical path lengths of reference arm 42 and sample arm 40. Connectors 210 are used at each end of assembly 200 to incorporate this fiber segment with rest of the system. In practice, it is difficult to precisely size the length of fiber segment 202. However, in scanner fabrication, it is feasible to configure a number of optical fiber segments 202 of similar lengths and find one “best fit” match for a particular scanner.

FIG. 10B shows an embodiment that can be more accurate than that shown in FIG. 10A, more suitable for shorter optical path length dimensions. A fiber ferrule 220 has an embedded optical fiber segment 222 that provides an optical path length with accuracy to within a few microns, obtained by polishing the end of the fiber-ferrule assembly. A hybrid fiber adapter 160 can be formed using a combination of optical fiber segments 202 of FIG. 10A with ferrule 220 of FIG. 10B.

FIG. 10C shows an optical fiber assembly 230 that has two smaller ferrules 232, each with embedded optical fiber segment 234. The two ferrules 232 are connected within a tube or sleeve 236. During fabrication, the ferrules 232 are first fixed to sleeve 236 with an appropriate separation so that the optical fiber segment 234 inserted into both ferrules can match the optical path lengths. As necessary, the overall length of the assembly 230 can be adjusted by changing the distance between ferrules 232 and the optical fiber 234 length, or by polishing the end of the assembly 230 to achieve the needed optical path length.

FIG. 10D shows how tapering can be used for lengthening the optical path length in small increments. In the tapering process, the ends of the fiber segment 202 are clamped and the fiber held taut. Then the clamped ends can be slowly pulled apart while the center section is heated, extending the fiber length. Fiber tapering can be performed on any fiber segment in sampling arm 40 or reference arm 42, with or without fiber adapter 160. The OCT signal can be monitored in real time during the tapering process in order to help fine-tune the stretching process that changes the OPD.

FIG. 10E shows the use of a tool for tapering to stretch fiber segment 202 itself, without ferrules or connectors. Alternately, any fiber segment can be stretched by a mechanical fiber stretcher, as shown in FIG. 10F.

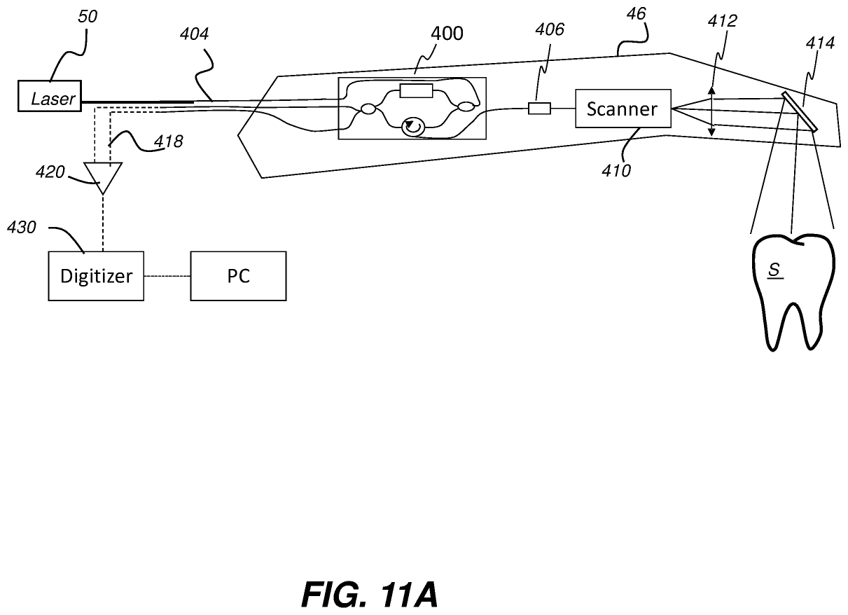

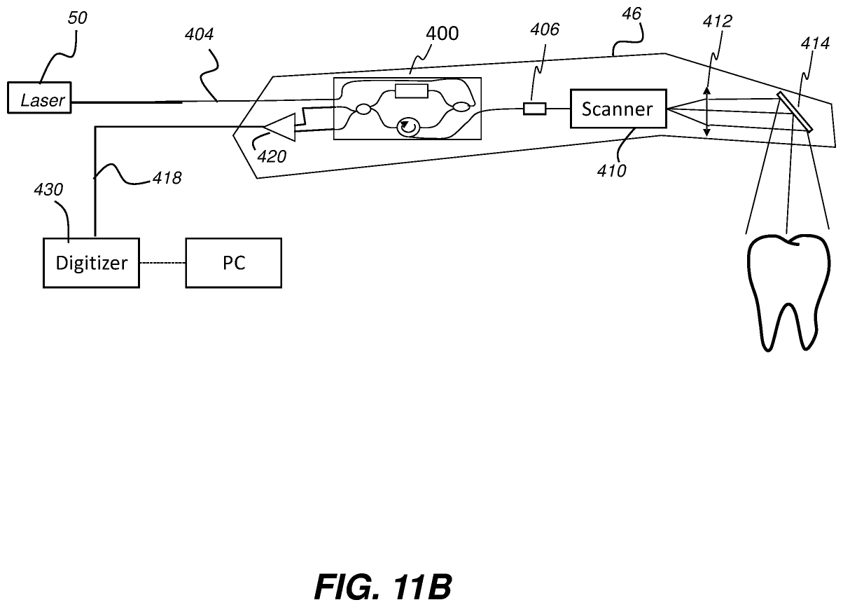

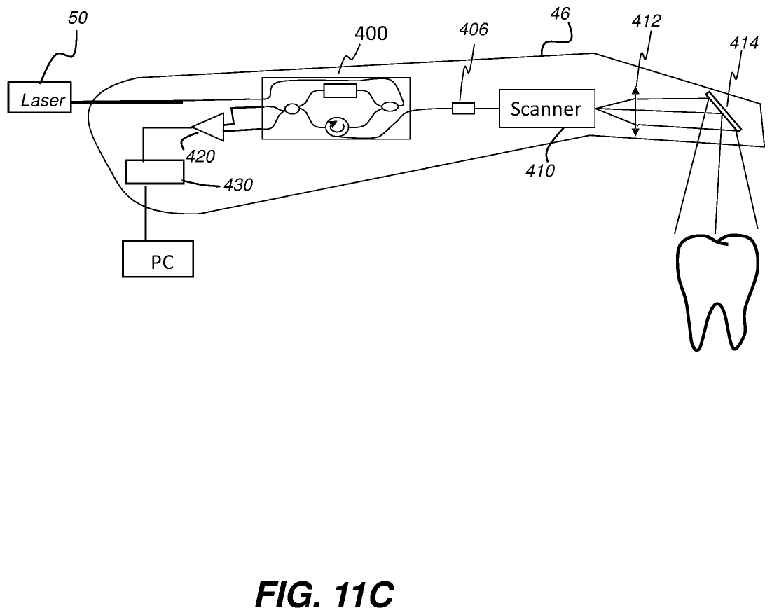

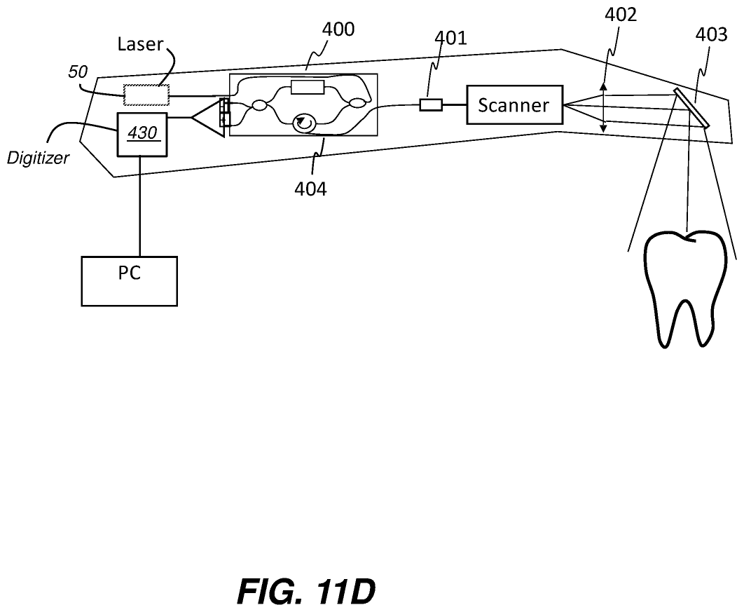

FIGS. 11A, 11B, 11C, and 11D show different configurations of probe 46 that include fiber interferometry system 400. In each of the embodiments shown, the compact fiber interferometry system 400, having fewer optical components and shorter fiber length than with conventional applications, can be fully housed within the handpiece, probe 46. The compact fiber interferometry system 400 can consist of a single interferometer for a single-channel OCT scanner, or multiple interferometers for a multi-channel OCT scanner, as shown in FIG. 9. An auxiliary Mach-Zehnder interferometer (MZI) installed either inside or outside of the probe (not shown) can be used to generate the k-clock signal or as a reference signal for numerical k-space resampling. Either MZI or fiber interferometry system 400 can be integrated on planar lightwave circuits (PLC), that could further reduce the size.

In FIG. 11A, swept source laser 50 input is generated external to handpiece probe 46 and conveyed to probe 46 through an optical cable 404. The output of the sampling arm is conveyed to a fiber collimator 406. The collimated output light then goes to a scanner 410 and through a focusing lens 412 to a folding mirror 414 that redirects the scanning beam to the tooth or other sample S. The scattered light returned from the sample S is conveyed as sampled light to interferometry system 400. Output signals are guided through a cable 418 and to a balanced detector 420. Detector 420 results are then provided to a digitizer 830 and to a computer PC for reconstruction and display.

FIG. 11B shows a more compact OCT system, wherein probe 46 also houses balanced detector 420. For a multi-channel OCT scanner system, balanced detector 420 consists of an array of balanced detectors, each receiving the output signal from each interferometer. Communication between detector 420 and external digitizer 430 can be wired, over cable 418 as shown, or wireless. Image quality is not affected by fiber bending, vibration, stretching, or other handling. Thus, using the FIG. 11B design, with balanced detector 420 and fiber system 400 packaged within probe 46, the number of cables connected to the handpiece is reduced along with the size of the optical engine.

FIG. 11C shows an embodiment with digitizer 430 housed within probe 46. Communication between probe 46 and the external PC can be wired or wireless.

FIG. 11D shows an embodiment with both digitizer 430 and laser 50 both housed within probe 46, along with other components for signal acquisition and processing. Communication between probe 46 and the external PC can be wired or wireless.

It can be observed that the succession of designs shown in FIGS. 11A-11D can help to reduce the number of cables required to the handpiece and can further reduce the overall size of the optical engine and improve system stability.

The invention has been described in detail with particular reference to a presently understood example embodiments, but it will be understood that variations and modifications can be affected within the spirit and scope of the disclosure.

For example, control logic processor 70 can be any of a number of types of logic processing device, including a computer or computer workstation, a dedicated host processor, a microprocessor, logic array, or other device that executes stored program logic instructions. The interferometer that is used for one or more channels, described in the example configurations given hereinabove as a type of Mach-Zehnder interferometer, can alternately be another appropriate type, such as a Michelson interferometer, for example, with appropriate component re-arrangement.

The presently disclosed example embodiments are therefore considered in all respects to be illustrative and not restrictive. The scope of the disclosure is indicated by the appended claims, and all changes that come within the meaning and range of equivalents thereof are intended to be embraced therein.

Consistent with at least one example embodiment, example methods/apparatus can use a computer program with stored instructions that perform on image data that is accessed from an electronic memory. As can be appreciated by those skilled in the image processing arts, a computer program of an example embodiment herein can be utilized by a suitable, general-purpose computer system, such as a personal computer or workstation. However, many other types of computer systems can be used to execute the computer program of described example embodiments, including an arrangement of one or networked processors, for example.

A computer program for performing methods of certain example embodiments described herein may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. Computer programs for performing example methods of described embodiments may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the application, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that can be directly associated with a display device and is periodically refreshed as needed in order to provide displayed data. This temporary storage buffer can also be considered to be a memory, as the term is used in the application. Memory is also used as the data workspace for executing and storing intermediate and final results of calculations and other processing. Computer-accessible memory can be volatile, non-volatile, or a hybrid combination of volatile and non-volatile types.

It will be understood that computer program products for example embodiments herein may make use of various image manipulation algorithms and/or processes that are well known. It will be further understood that example computer program product embodiments herein may embody algorithms and/or processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the application, are not specifically shown or described herein and may be selected from such algorithms, systems, hardware, components and elements known in the art.

Example embodiments according to the application can include various features described herein (individually or in combination).

While the invention has been illustrated with respect to one or more implementations, alterations and/or modifications can be made to the illustrated examples without departing from the spirit and scope of the appended claims. In addition, while a particular feature of the invention can have been disclosed with respect to only one of several implementations/example embodiments, such feature can be combined with one or more other features of the other implementations/example embodiments as can be desired and advantageous for any given or particular function.

The term “a” or “at least one of” is used to mean one or more of the listed items can be selected. The term “about” indicates that the value listed can be somewhat altered, as long as the alteration does not result in nonconformance of the process or structure to the illustrated example embodiment.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An optical coherence tomography scanner for imaging a sample, the optical coherence tomography scanner comprising:
a) a wavelength-tunable light source configured to generate scanning light having a range of wavelengths;
b) a scanning probe having a scanning head and one or more optical channels that convey light to and from the scanning head, each of the one or more optical channels comprising:
(i) a sample arm comprising optical fibers for conveying the scanning light to the sample and conveying scattered and back-reflected light from the sample to a detector,
(ii) a reference arm comprising optical fibers for conveying reference light from the wavelength-tunable light source,
(iii) an optical fiber or fiber system that defines an optical path distance for the sample arm or the reference arm,
(iv) a detector that generates an output signal according to combined light from the sample arm and the conveyed reference light; and
c) a digitizer that is energizable to generate digital data according to the detector output signal and to communicate the generated digital data to a computer for storage or display.

2. The optical coherence tomography scanner of claim 1, wherein the scanning head is configured for intraoral scanning.

3. The optical coherence tomography scanner of claim 1, wherein the sample arm, the reference arm, and the optical fiber or fiber system are housed within the scanning probe.

4. The optical coherence tomography scanner of claim 3, wherein the detector is housed within the scanning probe.

5. The optical coherence tomography scanner of claim 3, wherein the scanning probe is in a housing and the wavelength-tunable light source is housed within the housing.

6. The optical coherence tomography scanner of claim 1, wherein the scanning probe is a hand-held probe.

7. A method for acquiring an optical coherence tomography signal, the method comprising:
directing light from a wavelength-tunable light source to:
(i) a sample arm comprising optical fibers that are configured to convey the light to a sample and to convey scattered and back-reflected light returned from the sample to a detector,
(ii) a reference arm comprising optical fibers for conveying reference light from the wavelength-tunable light source, and
(iii) a fiber system that defines an optical path distance for the sample arm or the reference arm;
generating a signal according to interference between the returned light from the sample arm and from the reference arm;
generating digital data according to a detector output signal from the detector and communicating the generated digital data to a computer; and
storing or displaying an optical coherence tomography image formed according to the generated digital data.

8. The method of claim 7, wherein the fiber system comprises one or more fiber adapters, wherein the one or more fiber adapters are one or more pre fabricated fiber adapters or one or more adjustable fiber adapters used to match the optical path distance of the reference arm to the sample arm.

9. The method of claim 8, wherein the one or more fiber adapters are one or more optical fibers with specific optical path lengths.

10. The method of claim 8, wherein the one or more fiber adapters comprise a ferrule with an embedded optical fiber segment.

11. The method of claim 8, wherein the one or more fiber adapters comprise two or more ferrules and a connected tube or sleeve.

12. The method of claim 8, wherein the one or more fiber adapters comprise a fiber stretcher.

13. The method of claim 9, wherein at least one of the one or more fiber adapters is tapered to adjust for optical path length.

14. The method of claim 7, wherein the sample comprises an intraoral feature.
